(19) United States
(12) Patent Application Publication
(10) Pub. No.: US 2024/0172975 A1
(43) Pub. Date: May 30, 2024

(54) Flexible Capillary Tube with Integrated Luer Cap

(71) Applicant: Becton, Dickinson and Company, Franklin Lakes, NJ (US)

(72) Inventors: Shishir Prasad, Ramsey, NJ (US); Prasad Govindaraj, Coimbatore (IN); Balaji Kannan, Chennai (IN); Sridhaar Nandakumar, Chennai (IN)

(21) Appl. No.: 18/070,916

(22) Filed: Nov. 29, 2022

(51) Int. Cl. A61B 5/15

(52) U.S. Cl. CPC A61B 5/150992; A61B 5/150099; A61B 5/150389; A61M 25/0097

(57) ABSTRACT

A medical device includes a luer cap, a needle housed in the luer cap, a luer tube in fluid communication with the needle, an actuator coupled at least one of the needle and the tube, a spring operatively associated with the actuator, and a vacuum tube for blood sample collection.

### BACKGROUND OF THE INVENTION

### Field of the Invention

The present disclosure relates generally to blood collection devices and, more particularly, to flexible capillary tubes with integrated luer caps.

### Description of Related Art

Patients with intravenous (IV) catheters may require blood testing for determining the presence of infection, such as sepsis, or other for collecting other information about the patient. Sepsis commonly causes widespread inflammation (swelling) in the body, inflammation, and blood clotting during sepsis commonly causes reduced blood flow to limbs and vital organs, which can lead to organ failure and even death. So it is important to detect it as soon as possible. Sepsis occurs when germs get into a person's body. Bacterial infections cause most cases of sepsis. Patients requiring IV catheters commonly have suppressed immune systems, so it is important to limit contamination as much as possible. Typically, when a patient needs blood drawn, a needle and syringe are used to draw the blood directly from the patient or an external syringe is used to draw blood from the catheter port. One drawback to drawing blood directly from a catheter port using a syringe is a high chance of contamination in the catheter port.

Accordingly, those skilled in the art continue research and development efforts in the field of drawing blood through existing catheter ports.

### SUMMARY OF THE INVENTION

Disclosed is a medical device.

In one example, the medical device includes a luer cap, a needle housed in the luer cap, a luer tube in fluid communication with the needle, an actuator coupled at least one of the needle and the tube, and a spring operatively associated with the actuator.

The needle may be removably coupleable with the luer tube. The luer cap may include an inside surface and wherein the inside surface may be threaded.

The medical device may further include a vacuum collection tube removably coupled to the luer cap. The vacuum collection tube may define a vacuum chamber. The vacuum collection tube may include a distal end, a proximal end opposed from the distal end, a scaling cap positioned over the proximal end, and a septum coupled to the sealing cap.

The sealing cap may include an outside surface, the outside surface comprising a plurality of ribs. The needle may be movable between at least a first position and a second position. In the first position, the needle may be located below the septum and in the second position, the needle may be pierced through the septum and at least partially received within the vacuum chamber. Upon piercing through the septum, a vacuum may be drawn from the luer tube toward the vacuum chamber.

Also disclosed is a system for drawing blood from a patient.

In one example, the system includes a catheter hub, a catheter tube in fluid communication with the catheter hub, and a medical device removably coupleable to a portion of the catheter hub. The medical device includes a luer cap, a needle housed in the luer cap, a luer tube in fluid communication with the needle, an actuator coupled at least one of the needle and the tube, a spring operatively associated with the actuator, a vacuum collection tube removably coupled to the luer cap, the vacuum collection tube defining a vacuum chamber.

The luer tube may be removably coupleable with the catheter hub such that they are in fluid communication. The luer tube may be removably coupleable with the catheter tube such that they are in fluid communication. The luer tube may nest in the catheter tube. The medical device may be threadedly engageable with a portion of the catheter hub. The vacuum collection tube may include a distal end, a proximal end opposed from the distal end, a sealing cap positioned over the proximal end, and a septum coupled to the sealing cap. The sealing cap may be removably coupleable with the luer cap.

The needle may be movable between at least a first position, wherein the needle may be located below the septum, and a second position, wherein the needle may be pierced through the septum and at least partially received within the vacuum chamber. The actuator may facilitate movement of the needle from the first position and the second position, and wherein the spring facilitates movement of the needle and actuator from the second position to the first position. Upon piercing through the septum, a vacuum may be drawn from catheter tube toward the vacuum chamber to draw a blood sample from the patient.

Also disclosed is a method for testing a blood sample drawn through a catheter tube into a medical device including a luer cap, a needle housed in the luer cap, a luer tube in fluid communication with the needle, an actuator coupled at least one of the needle and the tube, a spring operatively associated with the actuator, and a vacuum collection tube coupled to the luer cap.

In one example, the method includes coupling the medical device with the catheter tube such that they are in fluid communication, actuating the actuator from a first position to a second position, drawing the blood sample into the vacuum collection tube, uncoupling the medical device from the catheter tube, and testing the blood sample.

The coupling may include coupling the medical device with a catheter hub in fluid communication with the catheter tube. The coupling and actuating occur concurrently. The method may further include transmitting data collected during the testing to a medical management system.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is an exploded schematic perspective view of a medical device;

FIG. 2 is a perspective schematic view of the medical device of FIG. 1;

FIG. 3 is a side schematic of a system for drawing blood from a patient;

FIG. 4 is a cross-sectional schematic side view of the system of FIG. 3;

FIG. 5 is a cross-sectional schematic side view of the system of FIG. 3;

FIG. 6 is a cross-sectional schematic side view of a system for drawing blood from a patient;

FIG. 7A is a cross-sectional schematic side view of a portion of the system of FIG. 3;

FIG. 7B is a cross-sectional schematic side view of a portion of the system of FIG. 3;

FIG. 8 is a cross-sectional schematic prospective view of a portion of the system of FIG. 3;

FIG. 9 is a cross-sectional schematic prospective view of a portion of the system of FIG. 3;

FIG. 10 is a flow chart of a method for testing a blood sample; and

FIG. 11 is a schematic diagram of a system for testing a blood sample.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The disclosed is a solution for streamlining detection of blood conditions, such as sepsis or other infection. The following are the signs and symptoms of sepsis: shortness of breath, high heart rate, fever, or shivering, or feeling very cold, severe muscle pain etc. The three stages of sepsis are: sepsis, severe sepsis, and septic shock. When a patient's immune system goes into overdrive in response to an infection, sepsis may develop as a result.

Sepsis is often diagnosed by simple measurements such temperature, heart rate and breathing rate, and blood testing. Other tests can help determine the type of infection, where it's located and which body functions have been affected. The following different biomarkers used in sepsis identification are CRP (C-reactive protein), PCT (Procalcitonin), Interleukin (IL)-6, Presepsin are the biomarkers that have been studied most frequently. Based on the research it has been identified that Interleukin (IL)-6 biomarker will be the effective and accurate for detection of the sepsis infection. These Interleukin (IL)-6 acts as both a pro-inflammatory and anti-inflammatory cytokine. The blood samples will be used as a matrix for identifying biomarkers is widely used in the detection of Sepsis.

The disclosure allows for extracting pure/fresh blood samples from deep in the Intravenous line through the catheter port by modifying existing CCMD products, such as the BD PureHub Disinfection Unit (DU) Cap. The disclosure integrates flexible capillary tubes and vacuum blood collection tubes with a luer cap (Modified BD PureHub Cap) that can be used to extract blood from patients, which will then be used to analyze the detection of sepsis.

At the proximal end of the Luer cap is a small hole and a circular rubber septum is positioned at the circumference of the hole. At the top of luer cap a vacuum blood collection tube may be fixed, which can be used to collect the fresh blood samples. The rubber septum helps to maintain a vacuum inside the blood collection tube by restricting the entry of outer atmosphere air. The one end of the flexible capillary tube is attached to the hallow bevel needle, the other end will be inserted into the vein for blood draw. At the proximal end of thread a free space houses an actuator and spring. The actuator may have lug design, which is seated on the circumference of the last thread profile and inside the actuator the spring were assembled, which will provide a cushioning effect to actuator when it get compressed against the catheter port. The flexible capillary tube is attached in fluid path communication with the needle, which is assembled with the actuator through, for example, interference fit.

In the proposed clinical state after catheterization, the luer cap Integrated with flexible capillary tube and vacuum blood collection tube are removably coupleable to a peripheral IV catheter through a threaded connection to collect a rich blood samples from vein. The flexible capillary tube may be advanced through the PIVC line, and may extend beyond the catheter tip, and enter the vessel to collect a fresh/pure blood sample. This flexible capillary tube is designed to extend beyond the suboptimal draw conditions around the IV to reach vessel locations where blood flow is optimal for aspiration. Once the luer cap is fully threaded with catheter port, the actuator will compress against catheter port, which in turn make the needle to pierce the septum and reaches the vacuumed blood collection tube. Due to the capillary and/or vacuum effect, the blood from the vein (high pressure area) will be traveling to the vacuum blood collection tube (low pressure area) through the flexible capillary tube. Once a desired mL of blood is collected in the blood collection tube, the threaded connection can be removed from the catheter port, which in turn makes the actuator enables the needle to travel back to its original position. When the needle returns to its original position, the septum becomes closed. Once the blood has been collected in the vacuum blood collection tube, the snaps will be separated from the luer cap, and the vacuum blood collection tube will be taken out separately. Blood into multiple vacuum tubes can be collected by attaching new tube at the back of the luer cap. The sealing cap keeps the blood sample in the vacuum tube free of external contaminant. Alternatively, the primary sealing cap can be removed from the vacuum tube, and a secondary sealing cap can replace it to close the blood collection tube and keep in the blood centrifuge machine for further blood test/blood culture evaluation.

The disclosure utilizes a vacuum blood collection tube and flexible capillary tube integrated with the currently commercially available Catheter Care disinfectant unit (DU) cap profile, which can be inserted into a Y port adapter of an IV catheter. Once the DU cap is inserted the pathway to the vacuum blood collection tube opens and the blood can flow from vein to the vacuum blood collection tube through the flexible capillary tube (blood starts flowing from high pressure area to low pressure area). This device can be assembled with any open IV catheter and can be assembled with any closed catheters through the side or Y ports.

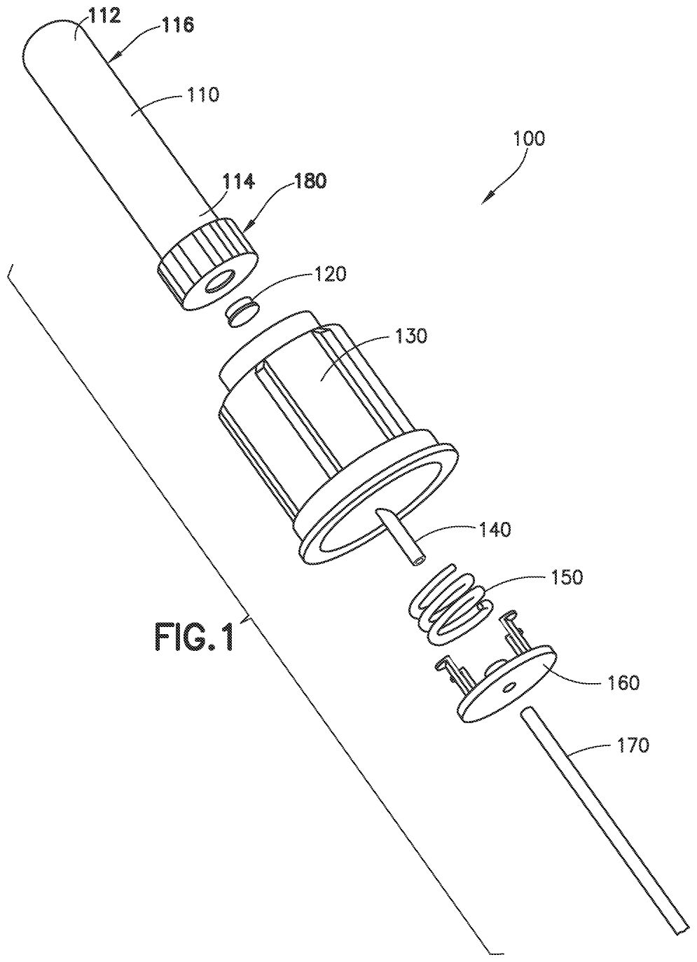

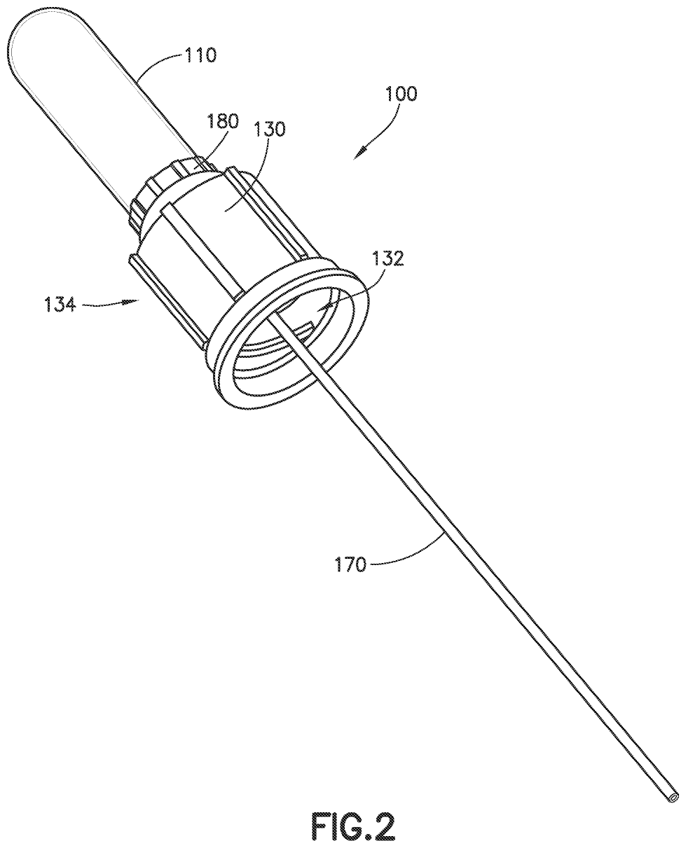

Referring to FIGS. 1 and 2, disclosed is a medical device 100. The medical device 100 includes a luer cap 130. The luer cap 130 comprises an inside surface 132. In one example, the inside surface 132 is threaded. The inside surface 132 may be threaded for threadedly engaging with other components of the medical device 100 and/or of a system 200 as shown and described below. Other means of engagement are contemplated and may be implemented, such as a snap engagement or a sliding engagement without limiting the scope of the disclosure.

The medical device 100 further includes a needle 140 housed in the luer cap 130. The needle 140 is movable between at least a first position and a second position, as described below.

Still referring to FIGS. 1 and 2, the medical device 100 includes a luer tube 170 in fluid communication with the needle 140. The luer tube 170 may be removably coupled with the needle 140 or may be integrated with the needle 140. In one example, the needle 140 is removably coupleable with the luer tube 170.

Still referring to FIG. 1, the medical device 100 includes an actuator 160 coupled at least one of the needle 140 and the luer tube 170. In one example, the actuator 140 is coupled to both the needle 140 and the luer tub 170. The actuator 160 is configured to facilitate movement of the needle 140 and/or the luer tube 170 between at least a first position and a second position. The actuator 160 may have a lug type of design and be seated on a circumference of the thread profile or inside surface 132 of the luer cap 130.

Still referring to FIG. 1, the medical device 100 includes a spring 150 operatively associated with the actuator 160. The spring 150 is positioned between the actuator 160 and a distal end 134 of the luer cap 130. The spring 150 is configured to compress when the actuator 160 moves the needle 140 to the second position and return to its original, decompressed, or relaxed state when the actuator 160 and needle 140 are in the first position. It is contemplated that other means of facilitating compression and decompression may be implemented without limiting the scope of the disclosure. For example, a foam material, such as a memory foam, or piston may be positioned between the actuator 160 and the distal end 134 of the luer cap 130 to facilitate movement of the actuator 160 and needle 140 rather than a spring 150.

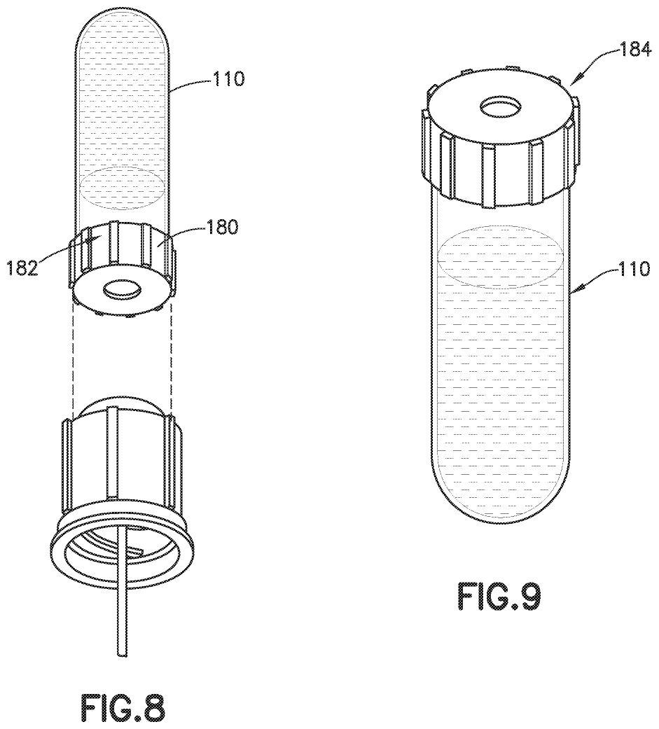

Still referring to FIGS. 1 and 2, the medical device 100 includes a vacuum collection tube 110 removably coupled to the luer cap 130. The vacuum collection tube 110 defines a vacuum chamber 116 configured to draw a vacuum for drawing a blood sample from a patient 230. In one example, the vacuum collection tube 110 includes a distal end 112 and a proximal end 114 opposed from the distal end 112. The vacuum collection tube 110 further includes a sealing cap 180 positioned over the proximal end 114. In one example, the vacuum collection tube 110 further includes a septum 120 coupled to the sealing cap 180. The sealing cap 180 may include an outside surface 182, and the outside surface 182 may include a plurality of ribs 184, sec FIGS. 8 and 9. The plurality of ribs 184 may be configured to engage with the luer cap 130 in a snap or nesting arrangement for securely coupling them together.

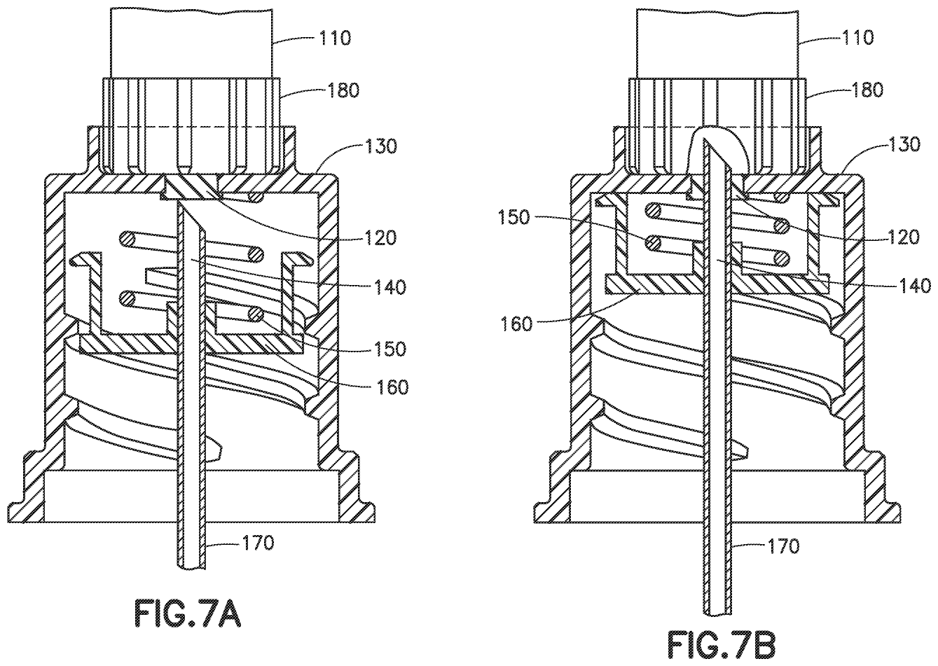

Referring to FIGS. 7a and 7b, in one or more examples, the needle 140, when in the first position, may be located below the septum 120 such that there is a gap between the needle 140 and the septum 120. When in the second position, the needle 140 may be pierced through at least a portion or through the entire thickness of the septum 120 and at least partially received within the vacuum chamber 112. Upon piercing through the septum 120, a vacuum is drawn from the luer tube 170 toward the vacuum chamber 112 to facilitate blood draw into the vacuum chamber 112. Due to a capillary effect, the blood from the vein (high pressure area) will travel to the vacuum collection tube 110 (low pressure area) through the luer tube 170 and needle 140 in fluid communication with the luer tube 170.

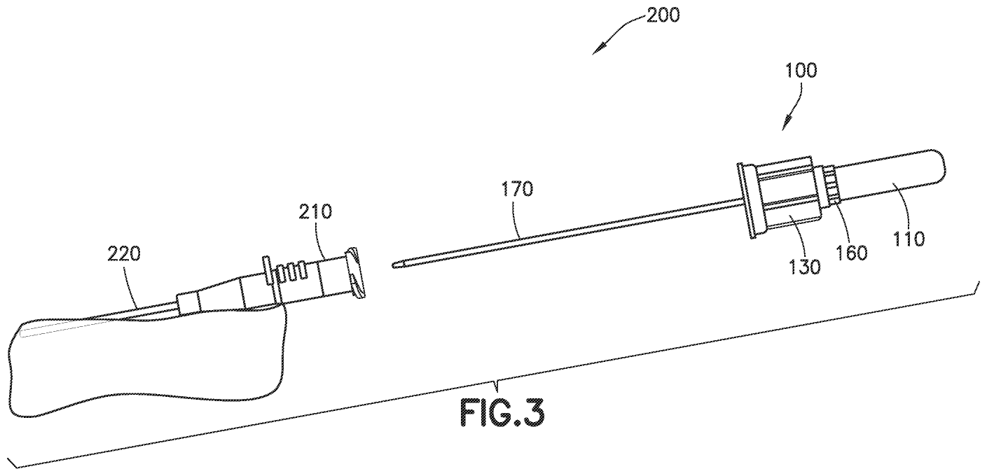

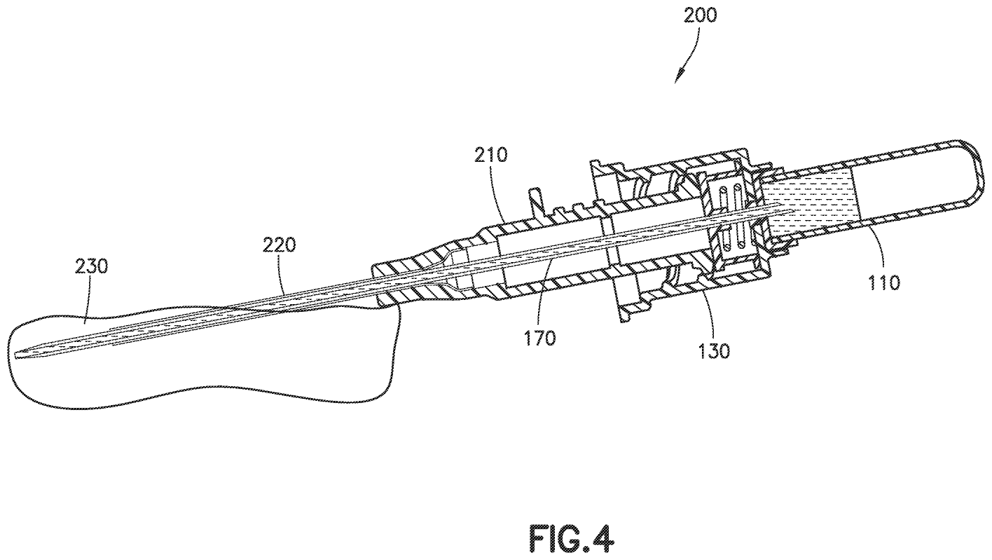

Referring to FIGS. 3-7B, also disclosed is a system 200 for drawing blood from a patient 230. The system 200 includes a catheter hub 210 and a catheter tube 220 in fluid communication with the catheter hub 210. The catheter tube 220 may be an intravenous (IV) catheter tube 220 subcutaneously insertable into a patient 230, such as a vein of a patient 230.

Still referring to FIGS. 3-7B, the system 200 includes a medical device 100 removably coupleable to a portion of the catheter hub 210 as shown and described herein such that they are in fluid communication. In one example, the medical device 100 is threadedly engageable with a portion of the catheter hub 210.

The medical device 100 includes a luer cap 130, a needle 140 housed in the luer cap 130, a luer tube 170 in fluid communication with the needle 140, an actuator 160 coupled at least one of the needle 140 and the luer tube 170, a spring 150 operatively associated with the actuator 160, and a vacuum collection tube 110 removably coupled to the luer cap 130, the vacuum collection tube 110 defining a vacuum chamber 116.

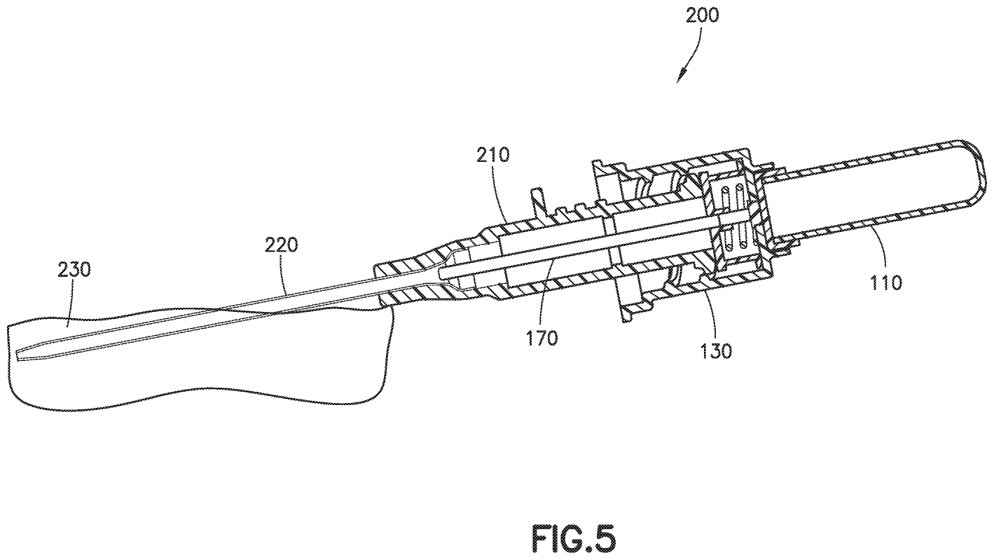

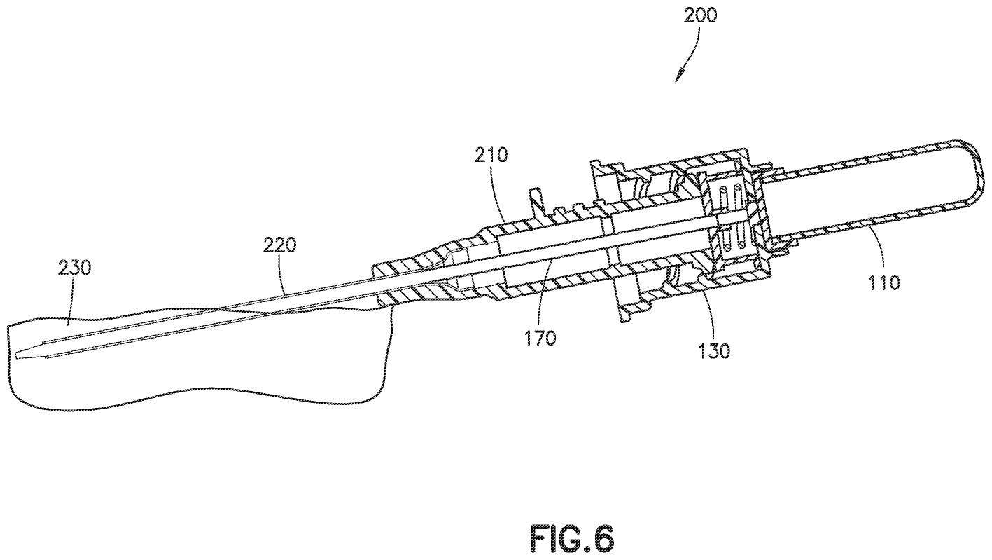

In one example, the luer tube 170 is removably coupleable with the catheter hub 210 such that they are in fluid communication, see FIG. 5. In another example, the luer tube 170 is removably coupleable with the catheter tube 220 such that they are in fluid communication, see FIG. 6. In one specific, non-limiting example, the luer tube 170 nests in the catheter tube 220. The luer tube 170 may be any length needed to accommodate the above-mentioned configurations. The luer tube may 170 extend into the patient 230, the catheter tube 220, or the catheter hub 210. The length of the luer tube 170 may be selected based on the clinician desire of from where the blood sample is required, e.g., from the catheter tube 220 or catheter hub 210, or from deep inside a vein of the patient 230, well beyond the catheter tube 220 or catheter hub 210. The luer tube 170 may be changed to collect blood from both a catheter component 210, 220, and from deep inside the vein of the patient 230, to compare the two collected samples. Drawing blood from different locations may be beneficial in ascertaining if the catheter tube 220 or catheter hub 210 is the source of infection, or if the infection is coming from within the patient 230.

The vacuum collection tube 110 includes a distal end 112, a proximal end 114 opposed from the distal end 112, a sealing cap 180 positioned over the proximal end 114, and a septum 120 coupled to the sealing cap 180. In one example, the sealing cap 180 of the vacuum collection tube 110 is removably coupleable with the luer cap 130.

The needle 140 of the medical device 100 is movable between at least a first position and a second position. The needle 140 is located below the septum 120 in the first position and the needle 140 is pierced through the septum 120 and at least partially received within the vacuum chamber 112 in the second position.

The actuator 160 of the system 200 facilitates movement of the needle 140 from the first position and the second position, and wherein the spring 140 facilitates movement of the needle 140 and actuator 160 from the second position to the first position. Upon piercing through the septum 120, a vacuum is drawn from catheter tube 220 toward the vacuum chamber 112 to draw a blood sample from the patient 230. Due to a capillary effect, the blood from the vein (high pressure area) will travel to the vacuum collection tube 110 (low pressure area) through the luer tube 170 and needle 140 in fluid communication with the luer tube 170.

After an adequate amount of blood has been drawn from the patient 230 to the vacuum collection tube 110, the vacuum collection tube 110 is configured to be uncoupled from the medical device 100 and can then be taken to another location for testing the blood sample, such as with a blood testing apparatus 250. In one example, the blood testing apparatus 250 is an electrochemical immunoassay.

In one example, after uncoupling from the medical device 100, the scaling cap 180 may be removed from the vacuum collection tube 110 and a secondary sealing cap 186 may replace the sealing cap 180 for a closed fit for transporting to other storage areas, such as a centrifuge machine, for further blood testing and blood culturing. The secondary sealing cap 186 may be a single, monolithic piece that does not have a septum 120 or other opening.

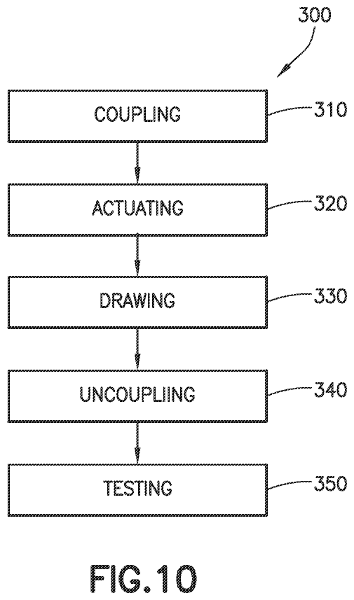

Referring to FIG. 10, disclosed is a method 300 for testing a blood sample drawn through a catheter tube 220 into a medical device 100 comprising a luer cap 130, a needle 140 housed in the luer cap 130, a luer tube 170 in fluid communication with the needle 140, an actuator 160 coupled at least one of the needle 140 and the luer tube 170, a spring 150 operatively associated with the actuator 160, and a vacuum collection tube 110 coupled to the luer cap 130,

Still referring to FIG. 10, the method 300 includes coupling 310 the medical device 100 with the catheter tube 220 such that they are in fluid communication. In one example, the coupling 310 comprises coupling the medical device 100 with a catheter hub 210 in fluid communication with the catheter tube 220. The coupling 310 may include sliding the medical device 100 over the catheter hub 210 that is in fluid communication with the catheter tube 220. In another example, the coupling 310 includes threadedly engaging the medical device 100 with the catheter hub 210 that is in fluid communication with the catheter tube 220

Still referring to FIG. 10, the method 300 includes actuating 320 the actuator 160 from a first position to a second position. In one example, the coupling 310 and actuating 320 occur concurrently. For example, upon positioning the medical device 100 in engagement with the catheter hub 210, the catheter hub 210 may push against the actuator 160 to actuate the actuator 160 in a direction toward the vacuum collection tube 110. Upon actuating 320 the actuator 160 from the first position to the second position, the spring 150 will compress and the needle 140 will pierce through the septum 120 of the vacuum collection tube 110.

Still referring to FIG. 10, the method 300 includes drawing 330 the blood sample into the vacuum collection tube 110. The drawing 330 will sequentially occur after the actuating 320 where the needle 140 becomes pierced through the septum 120 and a vacuum is drawn due to the pressure difference between the vacuum collection tube 110 and the patient 230, thus triggering the blood to flow through the catheter tube 220 to the vacuum collection tube 110 via the catheter hub 210 and/or the luer tube 170 into the needle 140 and then into the vacuum collection tube 110.

Still referring to FIG. 10, the method 300 includes uncoupling 340 the medical device 100 from the catheter tube 220. The uncoupling 340 may occur after a sufficient amount of blood has been drawn into the vacuum collection tube 110. The sufficient amount of blood may be a predetermined amount of blood. After the uncoupling 340, the catheter hub 210 will no longer be in abutment with the actuator 160. The spring 150 will return to its relaxed/decompressed position, which in turn pushes the actuator 160 and the needle 140 coupled with the actuator 160 back to the first position.

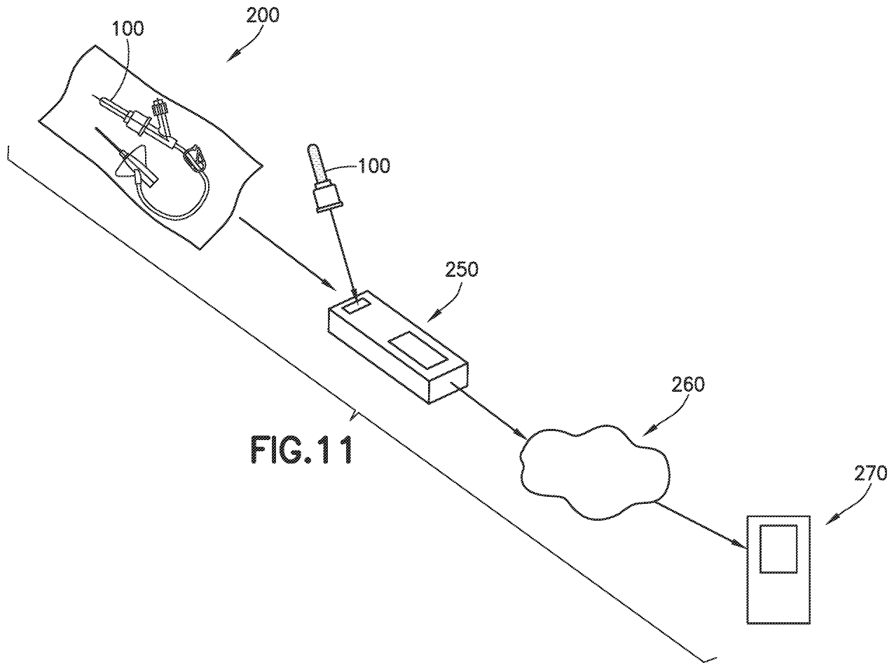

Still referring to FIG. 10, the method 300 includes testing 350 the blood sample. The testing 350 may be performed with a blood testing device 250, see FIG. 11. In one example, the blood testing device 250 is an electrochemical immunoassay. In one specific example, the collected blood sample will be placed in an electrode, which is coupled with an Optical Biosensor. Sensors will target the IL-6 biomarkers by identifying them and transfer the data to an IOT based device. In addition, the real time results/updates can be received at a nurse station using a preinstalled application in mobile/tablet. Time required for analysis is 20-25 minutes approx.

The method 300 may further include transmitting 360 data collected during the testing 350 to a medical management system 260. The blood testing device 250 may be in communication with the medical management system 260, such as wireless communication, to transmit the data for storage in a cloud or on a server, or for transmitting the data to a computer/phone for viewing and further analyzing the data collected from the testing 350.

Although non-limiting embodiments have been described in detail for the purpose of illustration and description, it is to be understood that such detail is solely for that purpose and that embodiments are not limited to the disclosed embodiments, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

The invention claimed is:

1. A medical device, comprising:
a luer cap;
a needle housed in the luer cap;
a luer tube in fluid communication with the needle;
an actuator coupled with at least one of the needle and the luer tube; and
a spring operatively associated with the actuator.

2. The medical device of claim 1, wherein the needle is removably coupleable with the luer tube.

3. The medical device of claim 1, wherein the luer cap comprises an inside surface and wherein the inside surface is threaded.

4. The medical device of claim 1, further comprising a vacuum collection tube removably coupled to the luer cap.

5. The medical device of claim 4, wherein the vacuum collection tube defines a vacuum chamber.

6. The medical device of claim 4, wherein the vacuum collection tube comprises:
a distal end;
a proximal end opposed from the distal end;
a sealing cap positioned over the proximal end; and
a septum coupled to the sealing cap.

7. The medical device of claim 6, wherein the sealing cap comprises an outside surface, the outside surface comprising a plurality of ribs.

8. The medical device of claim 6, wherein the needle is movable between at least a first position and a second position.

9. The medical device of claim 8, wherein, in the first position, the needle is located below the septum and in the second position, the needle is pierced through the septum and at least partially received within the vacuum chamber.

10. The medical device of claim 9, wherein, upon piercing through the septum, a vacuum is drawn from the luer tube toward the vacuum chamber.

11. A system for drawing blood from a patient, the system comprising:
a catheter hub;
a catheter tube in fluid communication with the catheter hub; and
a medical device removably coupleable to a portion of the catheter hub, the medical device comprising:
a luer cap;
a needle housed in the luer cap;
a luer tube in fluid communication with the needle;
an actuator coupled at least one of the needle and the luer tube;
a spring operatively associated with the actuator; and
a vacuum collection tube removably coupled to the luer cap, the vacuum collection tube defining a vacuum chamber.

12. The system of claim 11, wherein the luer tube is removably coupleable with the catheter hub such that they are in fluid communication.

13. The system of claim 11, wherein the luer tube is removably coupleable with the catheter tube such that they are in fluid communication.

14. The system of claim 11, wherein the luer tube extends into the catheter tube.

15. The system of claim 11, wherein the luer tube extends into the catheter hub.

16. The system of claim 11, wherein the medical device is threadedly engageable with a portion of the catheter hub.

17. The system of claim 11, wherein the vacuum collection tube comprises:
a distal end;
a proximal end opposed from the distal end;
a sealing cap positioned over the proximal end; and
a septum coupled to the sealing cap.

18. The system of claim 17, wherein the sealing cap is removably coupleable with the luer cap.

19. The system of claim 17, wherein the needle is movable between at least a first position wherein the needle is located below the septum and a second position wherein the needle is pierced through the septum and at least partially received within the vacuum chamber.

20. The system of claim 19, wherein the actuator facilitates movement of the needle from the first position and the second position, and wherein the spring facilitates movement of the needle and actuator from the second position to the first position.

21. The system of claim 19, wherein upon piercing through the septum, a vacuum is drawn from catheter tube toward the vacuum chamber to draw a blood sample from the patient.
